(19) United States
(12) Patent Application Publication
(10) Pub. No.: US 2017/0369845 A1
(43) Pub. Date: Dec. 28, 2017

(54) METHODS FOR ISOLATION OF PLATELETS

(71) Applicants: Manoochehr KHORSHIDI, San Diego, CA (US); ANTHROGENESIS CORPORATION, Warren, NJ (US)

(72) Inventor: Manoochehr KHORSHIDI, Great Neck, NY (US)

(73) Assignee: ANTHROGENESIS CORPORATION, Warren, NJ (US)

(21) Appl. No.: 15/541,354

(22) PCT Filed: Dec. 30, 2015
(86) PCT No.: PCT/US15/68044

§ 371 Date: Jun. 30, 2017

Related U.S. Application Data
(60) Provisional application No. 62/098,795, filed on Dec. 31, 2014.

(51) Int. Cl. C12N 5/078; A01N 1/02; A61K 35/19; C12N 5/00

(52) U.S. Cl. CPC C12N 5/0644; C12N 5/0087; A01N 1/0284; A01N 1/0289; A61K 35/19

(57) ABSTRACT

Provided herein are methods for the isolation of platelets, for example, isolation of platelets from umbilical cord blood. In certain embodiments, presented herein are methods for preparation of platelet rich plasma. In one aspect, provided herein are methods for isolation of platelets from blood. In certain embodiments, presented herein are methods for isolation of platelets from cord blood, e.g., human cord blood. The isolated platelets can be used for a variety of applications, including, for example, methods of wound healing, organ repair and/or regeneration, and/or tissue repair and/or regeneration, in either autologous or allogenic settings.

This application claims benefit of U.S. Provisional Patent Application No. 62/098,795, filed Dec. 31, 2014, the disclosure of which is incorporated by reference herein in its entirety.

### 1. FIELD

Provided herein are methods for isolation of platelets, for example, isolation of platelets from umbilical cord blood. In certain embodiments, the methods presented herein comprise preparation of platelet rich plasma (PRP).

### 2. BACKGROUND

Platelets are normal cellular components of blood. Although very small, platelets are known to contain various types of vesicles that carry a number of factors, e.g., growth factors, with potentially beneficial characteristics.

### 3. SUMMARY

In one aspect, provided herein are methods for isolation of platelets from blood. In certain embodiments, presented herein are methods for isolation of platelets from cord blood, e.g., human cord blood. The isolated platelets can be used for a variety of applications, including, for example, methods of wound healing, organ repair and/or regeneration, and/or tissue repair and/or regeneration, in either autologous or allogeneic settings.

In particular embodiments, platelets are separated from blood, for example cord blood, e.g., human cord blood, after erythrocyte removal from the blood. In specific embodiments, after erythrocyte removal, the resulting plasma is processed to separate the platelets in the plasma from other plasma components, for example, cellular components such as leukocytes.

In one embodiment, erythrocytes are removed from blood via centrigugation. In another embodiment, erythrocytes are removed from blood by utilizing a medium comprising components that result in erythrocyte sedimentation, either spontaneously or via centrifugation. In a particular embodiment, such a medium comprises a plasma volume expander, for example, hetastarch or pentastarch.

In one embodiment after erythrocyte removal from blood, for example cord blood, e.g., human cord blood, the resulting plasma is processed to enrich for the presence of platelets in the plasma, thereby producing platelet rich plasma (PRP). For example, plasma can be depleted for leukocytes, thereby enriching the platelet component of the plasma. In a specific embodiment, the plasma can be centrifuged, for example, centriguged at 200 to 500×G, e.g., 300-400×G, for a time sufficient to separate leukocytes from platelets in the plasma, for example, for 5, 10, 15, 20, 25, or 30 minutes, e.g., 10-30 minutes, 10-20 minutes, or 10-15 minutes. In such an embodiment, the resulting leukocyte-depleted plasma is platelet rich plasma (PRP).

In certain embodiments, prior to use or to storage, the PRP can be processed to yield a desired platelet concentration. In one embodiment, for example, the PRP can be centrifuged at 2000×G to 4000×G, e.g., 2000×G, for 10-20 minutes, e.g., for 15 minutes, pelleting and removing the resulting supernatant, to yield a desired PRP platelet concentration. In other embodiments, for example, the PRP can be centrifugued at 500×G to 2000×G for 20-60 minutes to yield a desired PRP platelet concentration.

In particular embodiments, platelets are isolated from blood, for example cord blood, e.g., human cord blood, after the blood has been processed to separate stem cells from the blood. In other particular embodiments, platelets can be isolated from blood, for example cord blood, e.g., human cord blood, without prior stem cell preservation. For example, blood, for example cord blood, e.g., human cord blood, can be processed to produce PRP by centrifugation, e.g., via 100-500 ×G, for example, 100-200×G, for 10-30 minutes, for example, 20-25 minutes. The resulting PRP can then be processed to pellet and remove the platelets from the remaining plasma.

In certain embodiments, the PRP is buffered prior to use. In another embodiment, the platelets in the PRP are separated from the remainder of the plasma, e.g., via centrifugation, and resuspended in a buffer prior to use.

In certain embodiments, the PRP is buffered prior to use. In another embodiment, the platelets in the PRP are separated from the remainder of the plasma, e.g., via centrifugation, and resuspended in a buffer prior to use.

In one embodiment, the PRP can be used immediately after generation. In certain embodiments, the PRP is buffered prior to use. In another embodiment, the platelets in the PRP are separated from the remainder of the plasma, e.g., via centrifugation, and resuspended in a buffer prior to use.

In yet another embodiment, the PRP can be stored for further use. For example, the PRP can be frozen or otherwise cryopreserved for further use. In other embodiments, the PRP can be freeze-dried for further use. For example, freeze-dried PRP can be cryopreserved. In another example, freeze-dried PRP can be stored at room temperature under vacuum.

In another embodiment, the platelets in the PRP are separated from the remainder of the plasma, e.g., via centrifugation, and resuspended in a buffer prior to storage. For example, the platelets in the PRP can separated from the remainder of the plasma, e.g., via centrifugation, and resuspended in a buffer prior to being frozen or otherwise cryopreserved for further use. In other embodiments, the platelets in the PRP are separated from the remainder of the plasma, e.g., via centrifugation, and resuspended in a buffer prior to being freeze-dried for further use. Freeze-dried platelets can, for example, be cryopreserved. In another example, freeze-dried platelets can be stored at room temperature under vacuum.

In certain embodiments, the PRP is buffered prior to storage. In another embodiment, the platelets in the PRP are separated from the remainder of the plasma, e.g., via centrifugation, and resuspended in a buffer suitable for storgage, e.g., cryopreservation, prior to storage.

In certain aspects, provided herein is a composition comprising the isolated PRP formulated to be administered to an individual, for example, administered by injection, e.g., local injection. In certain other aspects, provided herein is a composition comprising the isolated platelets formulated to be administered to an individual, for example, administered by injection, e.g., local injection.

In certain aspects, provided herein is a composition comprising the isolated PRP and stem cells, for example, placental stem cells (PDACs). In certain embodiments, such compositions are formulated to be administered to an individual, for example, administered by injection, e.g., local injection. In certain other aspects, provided herein is a composition comprising the isolated platelets and stem cells, for example, PDACs. In certain embodiments, such compositions are formulated to be administered to an individual, for example, administered by injection, e.g., local injection.

In some embodiments, the PRP and stem cells, e.g., placental stem cells, are combined to form said composition ex vivo prior to administration to, e.g., injection into, an individual. In other embodiments, the PRP is administered to, e.g., injected into, an individual in a first step, and the stem cells, e.g., placental stem cells, are administered to, e.g., injected into, the individual at or near the site of PRP administration in a second step, thereby forming the composition in vivo. In yet other embodiments, the stem cells, e.g., placental stem cells, are administered to, e.g., injected into, an individual in a first step, and the PRP is administered to, e.g., injected into, the individual at or near the site of stem cell administration in a second step, thereby forming the composition in vivo.

In other embodiments, the platelets and stem cells, e.g., placental stem cells, are combined to form said composition ex vivo prior to administration to, e.g., injection into, an individual. In other embodiments, the platelets are administered to, e.g., injected into, an individual in a first step, and the stem cells, e.g., placental stem cells, are administered to, e.g., injected into, the individual at or near the site of platelet administration in a second step, thereby forming the composition in vivo. In yet other embodiments, the stem cells, e.g., placental stem cells, are administered to, e.g., injected into, an individual in a first step, and the platelets are administered to, e.g., injected into, the individual at or near the site of stem cell administration in a second step, thereby forming the composition in vivo.

In a specific embodiment, said PDACs are CD10⁺, CD34⁻, CD105⁺, CD200⁺ placental stem cells. In another specific embodiment, said PDACs express CD200 and do not express HLA-G; or express CD73, CD105, and CD200; or express CD200 and OCT-4; or express CD73 and CD105 and do not express HLA-G. In yet other embodiments, said PDACs express one or more of CD44, CD90, HLA-A,B,C, or ABC-p, and/or do not express one or more of CD45, CD117, CD133, KDR, CD80, CD86, HLH-DR, SSEA3, SSE4, or CD38. In certain embodiments, the placental stem cells suppress the activity of an immune cell, e.g., suppress proliferation of a T cell.

In some embodiments, the volume to volume ratio of PRP to stem cells, e.g., placental stem cells, in the composition is between about 10:1 and 1:10. In some embodiments, the volume to volume ratio of PRP to stem cells, e.g., placental stem cells, in the composition is about 1:1. In some embodiments, the ratio of the number of platelets in the PRP to the number of stem cells, e.g., placental stem cells, is between about 100:1 and 1:100. In some embodiments, the ratio of the number of platelets in the PRP to the number of stem cells, e.g., placental stem cells, is about 1:1.

In certain aspects, provided herein is a composition comprising a matrix, hydrogel or scaffold, and the isolated PRP. In certain embodiments, such compositions are formulated to be administered to an individual. In certain other aspects, provided herein is a composition comprising a matrix, hydrogel or scaffold, and the isolated platelets. In certain embodiments, such compositions are formulated to be administered to an individual. In particular embodiments, such compositions comprise a natural matrix, e.g., a placental biomaterial such as an amniotic membrane material.

In certain aspects, provided herein is a composition comprising a matrix, hydrogel or scaffold, the isolated PRP and stem cells, for example, PDACs. In certain embodiments, such compositions are formulated to be administered to an individual. In certain other aspects, provided herein is a composition comprising a matrix, hydrogel or scaffold, the isolated platelets and stem cells, for example, PDACs. In certain embodiments, such compositions are formulated to be administered to an individual. In particular embodiments, such compositions comprise a natural matrix, e.g., a placental biomaterial such as an amniotic membrane material.

In some embodiments, the PRP of the compositions provided herein is autologous PRP. In some embodiments, the platelets of the compositions are autologous platelets. In some embodiments, the PRP of the compositions provided herein is allogeneic PRP. In some embodiments, the platelets of the compositions are allogeneic platelets.

In some embodiments, the PRP is derived from cord blood, e.g., human cord blood. In some embodiments, the platelets are derived from cord blood, e.g., human cord blood. In other embodiments, the PRP is derived from placental perfusate, e.g., human placental perfusate. In other embodiments, the platelets are derived from placental perfusate, e.g., human placental perfusate.

In particular aspects, the compositions are provided herein are for use in treating a disease, disorder or medical condition in an individual. For example, provided herein are methods of promoting wound healing comprising administering a composition provided herein to an individual in need of wound healing. In another example, provided herein are methods of promoting promoting tissue or organ repair or regeneration, comprising administering a composition provided herein to an individual in need of tissue or organ repair or regeneration. In a particular embodiment, provided herein are methods of bone repair or regeneration comprising administering a composition provided herein to an individual in need of bone repair or regeneration.

### 3.1 Definitions

As used herein, the term “about,” when referring to a stated numeric value, indicates a value within plus or minus 10% of the stated numeric value.

As used herein, the term “amount,” when referring to the placental stem cells described herein, means a particular number of placental cells.

As used herein, the term “stem cell” defines a cell that retains at least one attribute of a stem cell, e.g., a marker or gene expression profile associated with one or more types of stem cells; the ability to replicate at least 10-40 times in culture; multipotency, e.g., the ability to differentiate, either in vitro, in vivo or both, into cells of one or more of the three germ layers; the lack of adult (i.e., differentiated) cell characteristics, or the like.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “SH2” refers to an antibody that binds an epitope on the marker CD105. Thus, cells that are referred to as SH2⁺ are CD105⁺.

As used herein, the terms “SH3” and SH4” refer to antibodies that bind epitopes present on the marker CD73. Thus, cells that are referred to as SH3⁺ and/or SH4⁺ are CD73⁺.

As used herein, cells, e.g., PDACs are “isolated” if at least 50%, 60%, 70%, 80%, 90%, 95%, or at least 99% of other cells with which the stem cells are naturally associated are removed from the stem cells, e.g., during collection and/or culture of the stem cells.

As used herein, the term “isolated population of cells” means a population of cells that is substantially separated from other cells of the tissue, e.g., placenta, from which the population of cells is obtained or derived. In some embodiments, a population of, e.g., stem cells is “isolated” if at least 50%, 60%, 70%, 80%, 90%, 95%, or at least 99% of the cells with which the population of stem cells are naturally associated are removed from the population of stem cells, e.g., during collection and/or culture of the population of stem cells.

As used herein, the term “placental stem cell” refers to a stem cell or progenitor cell that is derived from, e.g., isolated from, a mammalian placenta, regardless of morphology, cell surface markers, or the number of passages after a primary culture, which adheres to a tissue culture substrate (e.g., tissue culture plastic or a fibronectin-coated tissue culture plate). The term “placenta stem cell” as used herein does not, however, refer to a trophoblast, a cytotrophoblast, embryonic germ cell, or embryonic stem cell, as those cells are understood by persons of skill in the art. The terms “placental stem cell” and “placenta-derived stem cell” may be used interchangeably. Unless otherwise noted herein, the term “placental” includes the umbilical cord. The placental stem cells disclosed herein are, in certain embodiments, multipotent in vitro (that is, the cells differentiate in vitro under differentiating conditions), multipotent in vivo (that is, the cells differentiate in vivo), or both.

As used herein, a stem cell is “positive” for a particular marker when that marker is detectable above background, e.g., by immunolocalization, e.g., by flow cytometry; or by RT-PCR, etc. For example, a cell or cell population is described as positive for, e.g., CD73 if CD73 is detectable on the cell, or in the cell population, in an amount detectably greater than background (in comparison to, e.g., an isotype control) or an experimental negative control for any given assay. In the context of, e.g., antibody-mediated detection, “positive,” as an indication a particular cell surface marker is present, means that the marker is detectable using an antibody, e.g., a fluorescently-labeled antibody, specific for that marker; “positive” also means that a cell or population of cells displays that marker in a amount that produces a signal, e.g., in a cytometer, ELISA, or the like, that is detectably above background. For example, a cell is “CD105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker compared to background. For example, a cell or population of cells is “CD34⁻” where the cell or population of cells is not detectably labeled with an antibody specific to CD34. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles. A cell is also positive for a marker when that marker can be used to distinguish the cell from at least one other cell type, or can be used to select or isolate the cell when present or expressed by the cell.

As used herein, “immunomodulation” and “immunomodulatory” mean causing, or having the capacity to cause, a detectable change in an immune response, and the ability to cause a detectable change in an immune response, either systemically or locally.

As used herein, “immunosuppression” and “immunosuppressive” mean causing, or having the capacity to cause, a detectable reduction in an immune response, and the ability to cause a detectable suppression of an immune response, either systemically or locally.

### 4. DETAILED DESCRIPTION

### 4.1 Methods of Obtaining Platelets and Platelet Rich Plasma

In one aspect, provided herein are methods for isolation of platelets from blood. In certain embodiments, presented herein are methods for isolation of platelets from cord blood, e.g., human cord blood, or placenta, e.g., human placenta, for example from placental perfusate.

The source of the platelets isolated using the methods described herein can be from any from a human or animal source of whole blood. For example, the PRP and isolated platelets may be prepared from an autologous source, an allogeneic source, a single source, or a pooled source of platelets and/or plasma, e.g., platelets harvested from corde blood, for example, human cord blood, or placenta, for example human placenta, e.g., from placental perfusate. For example, a donor that is to be a source of the blood used in the isolation methods presented herein can be a donor who has not been previously treated with a thrombolytic agent, such as heparin, tPA, or aspirin. In some embodiments, such a donor has not received a thrombolytic agent for at least 2 hours, 1 day, 2 weeks, or 1 month prior to withdrawing the blood.

In one embodiment, whole blood may be collected from a donor using a blood collection syringe. The amount of blood collected may depend on a number of factors, including, for example, the amount of platelets desired and the health of the donor. Any suitable amount of blood may be collected. For example, about 30 to 60 ml of whole blood may be drawn. In an exemplary embodiment, about 11 ml of blood may be withdrawn into a syringe that contains about 5 ml of an anticoagulant, such as acid-citrate-phosphate or citrate-phosphate-dextrose solution. The syringe may be attached to an apheresis needle, and primed with the anticoagulant. Blood may be drawn from the donor using standard aseptic practice. In some embodiments, a local anesthetic such as anbesol, benzocaine, lidocaine, procaine, bupivicaine, or any appropriate anesthetic known in the art may be used to anesthetize the insertion area.

In particular embodiments, the platelets are isolated from cord blood, e.g., human cord blood. Cord blood can be obtained using standard methods well known in the art.

In particular embodiments, platelets are isolated from placenta, e.g., human placenta, for example from placental perfusate. An exemplary method for isolation of placental perfusate is described below.

The placenta, for example, human placenta, e.g., human, full-term placenta, should be placed in a sterile, insulated container at room temperature and delivered to the laboratory within 4 hours of birth. The placenta is discarded if, on inspection, it has evidence of physical damage such as fragmentation of the organ or avulsion of umbilical vessels. Optionally, prior to such delivery, the placenta and any umbilical cord attached thereto can be exsanguinated or partially exsanguinated.

The placenta is maintained at room temperature (23°+/−2° C.) or refrigerated (4° C.) in sterile containers for 2 to 20 hours. Periodically, the placenta is immersed and washed in sterile saline at 25°+/−3° C. to remove any visible surface blood or debris. The umbilical cord is transected approximately 5 cm from its insertion into the placenta and the umbilical vessels are cannulated with Teflon or polypropylene catheters connected to a sterile fluid path allowing bidirectional perfusion of the placenta and recovery of the effluent fluid.

The placenta is maintained under conditions which simulate and sustain a physiologically compatible environment for the recruitment of cells. The cannula is flushed with IMDM serum-free medium (GibcoBRL, NY) containing 2U/ml heparin (Elkins-Sinn, N.J.). Perfusion of the placenta is performed at a rate of 50 mL per minute. During the course of the procedure, the placenta is gently massaged to aid in the perfusion process and assist in the recovery of cellular material. Effluent fluid is collected from the perfusion circuit by both gravity drainage and aspiration through the arterial cannula.

The perfusion and collection procedures may be repeated until the number of recovered nucleated cells falls below 100/microL. The perfusates are pooled and used to isolate platelets are described heriein.

In particular embodiments, platelets are separated from blood, for example cord blood, e.g., human cord blood, or placenta, e.g., human placenta, for example from placental perfusate, after erythrocyte removal from the blood. In specific embodiments, after erythrocyte removal, the resulting plasma is processed to separate the platelets in the plasma from other plasma components, for example, cellular components such as leukocytes.

In one embodiment, erythrocytes are removed from blood via centrigugation. In another embodiment, erythrocytes are removed from blood by utilizing a medium comprising components that result in erythrocyte sedimentation, either spontaneously or via centrifugation. In a particular embodiment, such a medium comprises a plasma volume expander, for example, hetastarch or pentastarch.

In one embodiment after erythrocyte removal from blood, for example cord blood, e.g., human cord blood, or placenta, e.g., human placenta, for example from placental perfusate,the resulting plasma is processed to enrich for the presence of platelets in the plasma, thereby producing platelet rich plasma (PRP). For example, plasma can be depleted for leukocytes, thereby enriching the platelet component of the plasma. In a specific embodiment, the plasma can be centrifuged, for example, centriguged at 200 to 500×G, e.g., 300-400×G, for a time sufficient to separate leukocytes from platelets in the plasma, for example, for 5, 10, 15, 20, 25, or 30 minutes, e.g., 10-30 minutes, 10-20 minutes, or 10-15 minutes. In such an embodiment, the resulting leukocyte-depleted plasma is platelet rich plasma (PRP).

In certain embodiments, prior to use or to storage, the PRP can be processed to yield a desired platelet concentration. In one embodiment, for example, the PRP can be centrifuged at 2000×G to 4000×G, e.g., 2000×G, for 10-20 minutes, e.g., for 15 minutes, to yield a desired PRP platelet concentration. In other embodiments, for example, the PRP can be centrifugued at 500×G to 2000×G for 20-60 minutes to yield a desired PRP platelet concentration.

In particular embodiments, platelets are isolated from blood, for example cord blood, e.g., human cord blood, or placenta, e.g., human placenta, for example from placental perfusate, after the blood has been processed to separate stem cells from the blood. In other particular embodiments, platelets can be isolated from blood, for example cord blood, e.g., human cord blood, or placenta, e.g., human placenta, for example from placental perfusate, without prior stem cell preservation. For example, blood, for example cord blood, e.g., human cord blood, or placenta, e.g., human placenta, for example from placental perfusate, can be processed to produce PRP by centrifugation, e.g., via 100-500×G, for example, 100-200×G, for 10-30 minutes, for example, 20-25 minutes. The resulting PRP can then be processed to pellet and remove the platelets from the remaining plasma.

In certain embodiments, the PRP is buffered prior to use. In another embodiment, the platelets in the PRP are separated from the remainder of the plasma, e.g., via centrifugation, and resuspended in a buffer prior to use.

In certain embodiments, the PRP is buffered prior to use. In another embodiment, the platelets in the PRP are separated from the remainder of the plasma, e.g., via centrifugation, and resuspended in a buffer prior to use.

In one embodiment, the PRP can be used immediately after generation. In certain embodiments, the PRP is buffered prior to use. In another embodiment, the platelets in the PRP are separated from the remainder of the plasma, e.g., via centrifugation, and resuspended in a buffer prior to use.

In certain embodiments, the PRP or resuspended platelets may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the PRP or ressuspended platelets to physiological pH between about 6.5 and about 8.0. In certain embodiments, the physiological pH may be adjusted to about pH 7.3 to about pH 7.5, and more specifically, about pH 7.4. In certain embodiments, the buffering agent may be an 8.4% sodium bicarbonate solution. In a particular embodiment, for each cc of PRP isolated from whole blood, 0.05 cc of 8.4% sodium bicarbonate may be added.

In yet another embodiment, the PRP can be stored for further use. For example, the PRP can be frozen or otherwise cryopreserved for further use. In a specific embodiment, a cryopreservative such as DMSO, glycerol, or EPILIFE™ Cell Freezing Medium (Cascade Biologics)) is added prior to freezing.

In other embodiments, the PRP can be freeze-dried for further use. For example, freeze-dried PRP can be cryopreserved. In another example, freeze-dried PRP can be stored at room temperature under vacuum.

In another embodiment, the platelets in the PRP are separated from the remainder of the plasma, e.g., via centrifugation, and resuspended in a buffer prior to storage. For example, the platelets in the PRP can separated from the remainder of the plasma, e.g., via centrifugation, and resuspended in a buffer prior to being frozen or otherwise cryopreserved for further use. In a specific embodiment, a cryopreservative such as DMSO, glycerol, or EPILIFE™ Cell Freezing Medium (Cascade Biologics)) is added prior to freezing.

In other embodiments, the platelets in the PRP are separated from the remainder of the plasma, e.g., via centrifugation, and resuspended in a buffer prior to being freeze-dried for further use. Freeze-dried platelets can, for example, be cryopreserved. In another example, freeze-dried platelets can be stored at room temperature under vacuum.

In certain embodiments, the PRP is buffered prior to storage. In another embodiment, the platelets in the PRP are separated from the remainder of the plasma, e.g., via centrifugation, and resuspended in a buffer suitable for storgage, e.g., cryopreservation, prior to storage.

### 4.2 Compositions Comprising Platelets and Platelet Rich Plasma

In certain aspects, provided herein is a composition comprising the isolated PRP obtained via the methods presented herein. In some embodiments, compositions provided herein comprise PRP which comprises platelet cells at a concentration of at least 1.1-fold greater than the concentration of platelets in whole blood, e.g., unprocessed whole blood, used to generate the PRP. In some embodiments, a composition provided herein comprises PRP that comprises platelet cells at a concentration of about 1.1-fold to about 10-fold greater than the concentration of platelets in whole blood, e.g., unprocessed whole blood, used to generate the PRP. In some embodiments, a composition provided herein comprises PRP that comprises platelet cells at a concentration of about 1.5, 2.0, 2.5, 3.0, 3.5, 4, 4.5, 5, 5.5, 6, 6.5, 7, 7.5, 8, 8.5, 9, 9.5, 10-fold, or more than 10-fold greater than the concentration of platelets in whole blood, e.g., unprocessed whole blood, used to generate the PRP.

In certain other aspects, provided herein is a composition comprising platelets obtained via the methods presented herein. In some embodiments, compositions provided herein comprise comprise platelet cells at a concentration of at least 1.1-fold greater than the concentration of platelets in whole blood, e.g., unprocessed whole blood, used to generate isolated platelets. In some embodiments, a composition provided herein comprises platelet cells at a concentration of about 1.1-fold to about 10-fold greater than the concentration of platelets in whole blood, e.g., unprocessed whole blood, used to generate the isolated platelets. In some embodiments, a composition provided herein comprises platelet cells at a concentration of about 1.5, 2.0, 2.5, 3.0, 3.5, 4, 4.5, 5, 5.5, 6, 6.5, 7, 7.5, 8, 8.5, 9, 9.5, 10-fold, or more than 10-fold greater than the concentration of platelets in whole blood, e.g., unprocessed whole blood, used to generate the isolated platelets.

Generally, a microliter of whole blood comprises between 140,000 and 500,000 platelets. In some embodiments, the platelet concentration in the compositions provided herein is between about 150,000 and about 2,000,000 platelets per microliter. In some embodiments, the platelet concentration in the compositions presented herein is about 150,000, 200,000, 300,000, 400,000, 500,000, 600,000, 700,000, 800,000, 900,000, 1,000,000, 1,100,000, 1,100,000, 1,200,000, 1,300,000, 1,400,000, 1,500,000, 1,600,000, 1,700,000, 1,800,000, 1,900,000, or 2,000,000 platelets per microliter. In some embodiments, the platelet concentration in the compositions presented herein is about 2,500,000 to about 5,000,000, or about 5,000,000 to about 7,000,000 platelets per microliter.

In certain aspects, provided herein is a composition comprising the isolated PRP formulated to be administered to an individual, for example, administered by injection, e.g., local injection. In certain other aspects, provided herein is a composition comprising the isolated platelets formulated to be administered to an individual, for example, administered by injection, e.g., local injection.

In certain aspects, provided herein is a composition comprising the isolated PRP and stem cells, for example, placental stem cells (PDACs). In certain embodiments, such compositions are formulated to be administered to an individual, for example, administered by injection, e.g., local injection. In certain other aspects, provided herein is a composition comprising the isolated platelets and stem cells, for example, PDACs. In certain embodiments, such compositions are formulated to be administered to an individual, for example, administered by injection, e.g., local injection.

In some embodiments, the PRP and stem cells, e.g., placental stem cells, are combined to form said composition ex vivo prior to administration to, e.g., injection into, an individual. In other embodiments, the PRP is administered to, e.g., injected into, an individual in a first step, and the stem cells, e.g., placental stem cells, are administered to, e.g., injected into, the individual at or near the site of PRP administration in a second step, thereby forming the composition in vivo. In yet other embodiments, the stem cells, e.g., placental stem cells, are administered to, e.g., injected into, an individual in a first step, and the PRP is administered to, e.g., injected into, the individual at or near the site of stem cell administration in a second step, thereby forming the composition in vivo.

In other embodiments, the platelets and stem cells, e.g., placental stem cells, are combined to form said composition ex vivo prior to administration to, e.g., injection into, an individual. In other embodiments, the platelets are administered to, e.g., injected into, an individual in a first step, and the stem cells, e.g., placental stem cells, are administered to, e.g., injected into, the individual at or near the site of platelet administration in a second step, thereby forming the composition in vivo. In yet other embodiments, the stem cells, e.g., placental stem cells, are administered to, e.g., injected into, an individual in a first step, and the platelets are administered to, e.g., injected into, the individual at or near the site of stem cell administration in a second step, thereby forming the composition in vivo.

Placental stem cells useful in the compositions and methods described herein are described herein and, e.g., in U.S. Pat. Nos. 7,311,904; 7,311,905; 7,468,276; 8,057,788; and 8,202,703, the disclosures of which are hereby incorporated by reference in their entireties.

In a specific embodiment, said PDACs are CD10⁺, CD34⁻, CD105⁺, CD200⁺ placental stem cells. In another specific embodiment, the CD10⁺, CD34⁻, CD105⁺, CD200⁺ placental stem cells are additionally CD45⁻ or CD90⁺. In another specific embodiment, such cells are additionally CD80⁻ and/or CD86−.

In certain embodiments, said placental stem cells are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ cells described above are additionally one or more of CD29⁺, CD38⁻, CD44⁺, CD54⁺, SH3⁺ or SH4⁺. In another specific embodiment, the cells are additionally CD44⁺. In another specific embodiment of any of the isolated CD34⁻, CD10⁺, CD105⁺ placental stem cells above, the cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34⁻, CD10⁺, CD105⁺ cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In another specific embodiment, any of the placental stem cells described herein are additionally ABC-p⁺, as detected by flow cytometry, or OCT-4⁺ (POU5F1⁺), as determined by reverse-transcriptase polymerase chain reaction (RT-PCR), wherein ABC-p is a placenta-specific ABC transporter protein (also known as breast cancer resistance protein (BCRP) and as mitoxantrone resistance protein (MXR)), and OCT-4 is the Octamer-4 protein (POU5F1).

In another specific embodiment, any of the placental stem cells described herein are additionally SSEA3⁻ or SSEA4⁻, as determined by flow cytometry, wherein SSEA3 is Stage Specific Embryonic Antigen 3, and SSEA4 is Stage Specific Embryonic Antigen 4. In another specific embodiment, any of the placental stem cells described herein are additionally SSEA3⁻ and SSEA4⁻.

In another specific embodiment, any of the placental stem cells described herein are additionally one or more of MHC-I⁺ (e.g., HLA-A,B,C⁺), MHC-II⁻ (e.g., HLA-DP,DQ,DR⁻) or HLA-G⁻. In another specific embodiment, any of the placental stem cells described herein are additionally one or more of MHC-I⁺ (e.g., HLA-A,B,C⁺), MHC-II⁻ (e.g., HLA-DP,DQ,DR⁻) and HLA-G⁻.

In yet another specific embodiment, said PDACs express CD200 and do not express HLA-G; or express CD73, CD105, and CD200; or express CD200 and OCT-4; or express CD73 and CD105 and do not express HLA-G. In yet other embodiments, said PDACs express one or more of CD44, CD90, HLA-A,B,C, or ABC-p, and/or do not express one or more of CD45, CD117, CD133, KDR, CD80, CD86, HLH-DR, SSEA3, SSE4, or CD38. In certain embodiments, the placental stem cells suppress the activity of an immune cell, e.g., suppress proliferation of a T cell.

In some embodiments, the volume to volume ratio of PRP to stem cells, e.g., placental stem cells, in the composition is between about 10:1 and 1:10. In some embodiments, the volume to volume ratio of PRP to stem cells, e.g., placental stem cells, in the composition is about 1:1. In some embodiments, the ratio of the number of platelets in the PRP to the number of stem cells, e.g., placental stem cells, is between about 100:1 and 1:100. In some embodiments, the ratio of the number of platelets in the PRP to the number of stem cells, e.g., placental stem cells,is about 1:1.

In some embodiments, the volume to volume ratio of stem cells, e.g., placental stem cells, to PRP is about 10:1, 9.5:1, 9:1, 8.5:1, 8:1, 7.5:1, 7:1, 6.5:1, 6:1, 5.5.:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1.9.5, or 1:10. In some embodiments, the volume to volume ratio of stem cells, e.g., placental stem cells, to PRP is about 100:1, 95:1, 90:1, 85:1, 80:1, 75:1, 70:1, 65:1, 60:1, 55.:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1;70, 1:75, 1:80, 1:85, 1:90, 1.95, or 1:100. In particular embodiments, the ratio of the number of stem cells, e.g., placental stem cells, to the number of platelets in the PRP is about 100:1, 95:1, 90:1, 85:1, 80:1, 75:1, 70:1, 65:1, 60:1, 55.:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1;70, 1:75, 1:80, 1:85, 1:90, 1.95, or 1:100.

The compositions comprising stem cells, e.g., placental stem cells, and PRP or platelets provided herein can comprise a therapeutically-effective amount of stem cells, e.g., placental stem cells, or PRP or platelets, or both. The combination compositions can comprise at least 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ stem cells, e.g., placental stem cells, platelets, e.g., platelets in PRP, or both, or no more than 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or1×10¹¹ stem cells, e.g., placental stem cells, platelets, e.g., platelets in PRP, or both.

In one embodiment, such a composition comprises about 300 million stem cells, e.g., placental stem cells. In certain other embodiments, such a composition comprises a range from 1 million to 10 billion stem cells, e.g., placental stem cells, between 10 million and 1 billion stem cells, e.g., placental stem cells, or between 100 million and 500 million stem cells, e.g., placental stem cells.

In certain aspects, provided herein is a composition comprising a matrix, hydrogel or scaffold, and the isolated PRP. In certain embodiments, such compositions are formulated to be administered to an individual. In certain other aspects, provided herein is a composition comprising a matrix, hydrogel or scaffold, and the isolated platelets. In certain embodiments, such compositions are formulated to be administered to an individual. In particular embodiments, such compositions comprise a natural matrix, e.g., a placental biomaterial such as an amniotic membrane material.

In certain aspects, provided herein is a composition comprising a matrix, hydrogel or scaffold, the isolated PRP and stem cells, for example, PDACs. In certain embodiments, such compositions are formulated to be administered to an individual. In certain other aspects, provided herein is a composition comprising a matrix, hydrogel or scaffold, the isolated platelets and stem cells, for example, PDACs. In certain embodiments, such compositions are formulated to be administered to an individual.

In particular embodiments, compositions presented herein comprise a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. In certain embodiments, placental biomaterials on which PRP or isolated platelets and, optionally, stem cells, e.g., placental stem cells, can be added are described in Hariri, U.S. Application Publication No. 2004/0048796, which is incorporated herein in its entirety. Additionally biomaterials on on which PRP or isolated platelets and, optionally, stem cells, e.g., placental stem cells, can be added are described in Hariri, U.S. Application Publication No. 2008/0181935, which is incorporated herein in its entirety.

In other embodiments, compositions presented herein comprise PRP or isolated platelets and, optionally, stem cells, e.g., placental stem cells, suspended in a hydrogel solution, for example, a hydrogel solution suitable for injection. Suitable hydrogels for such compositions include, for example, self-assembling peptides, such as RAD16. In one embodiment, a hydrogel solution comprising PRP or isolated platelets and, optionally, stem cells, e.g., placental stem cells, can be allowed to harden, for instance in a mold, to form a matrix for implantation. In embodiments comprising stem cells, e.g., placental stem cells, such a matrix can also be cultured so that the cells are mitotically expanded prior to implantation. In particular embodiments, the hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials can include, for example, polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, a composition presented herein comprises an in situ polymerizable gel (see., e.g., U.S. Patent Application Publication 2002/0022676; Anseth et al., J. Control Release, 78 (1-3):199-209 (2002); and Wang et al., Biomaterials, 24 (22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In certain embodiments, compositions presented herein comprise PRP or isolated platelets and, optionally, stem cells, e.g., placental stem cells, on a three-dimensional framework or scaffold, e.g., a three-dimensional framework or scafford suitable for implantation in vivo.

Examples of scaffolds that can be used in such compositions include, for example, nonwoven mats, porous foams, or self assembling peptides. Nonwoven mats can be formed, for example, using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds. Other scaffolds may, for example, comprise oxidized cellulose or oxidized regenerated cellulose.

In another embodiment, the scaffold is, or comprises, a nanofibrous scaffold, e.g., an electrospun nanofibrous scaffold. In a more specific embodiment, said nanofibrous scaffold comprises poly(L-lactic acid) (PLLA), type I collagen, a copolymer of vinylidene fluoride and trifluoroethylnee (PVDF-TrFE), poly(-caprolactone), poly(L-lactide-co-ε-caprolactone) [P(LLA-CL)] (e.g., 75:25), and/or a copolymer of poly(3-hydroxybutyrate-co-3-hydroxyvalerate) (PHBV) and type I collagen. In another more specific embodiment, said scaffold promotes the differentiation of placental stem cells into chondrocytes. Methods of producing nanofibrous scaffolds, e.g., electrospun nanofibrous scaffolds, are known in the art. See, e.g., Xu et al., Tissue Engineering 10(7):1160-1168 (2004); Xu et al., Biomaterials 25:877-886 (20040; Meng et al., J. Biomaterials Sci., Polymer Edition 18 (1):81-94 (2007).

In yet another embodiment, compositions presented herein comprise PRP or isolated platelets and, optionally, stem cells, e.g., placental stem cells, and a physiologically-acceptable ceramic material including, for example, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include, for example, SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, compositions presented herein comprise PRP or isolated platelets and, optionally, stem cells, e.g., placental stem cells, and a felt, which can be, e.g., composed of a multifilament yarn made from a bioabsorbable material such as PGA, PLA, PCL copolymers or blends, or hyaluronic acid.

In a particular embodiment, compositions presented herein comprise PRP or isolated platelets and, optionally, stem cells, e.g., placental stem cells, and a foam scaffold, e.g., a foam scaffold made of composite structures. Such foam scaffolds can, for example, be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inclusion of the PRP or isolated platelets and, optionally, stem cells, e.g., placental stem cells, to enhance cell attachment. External surfaces of a matrix may, for example, be modified to improve the attachment or growth of cells and, if desired, differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental stem cells.

In some embodiments, the PRP of the compositions provided herein is autologous PRP. In some embodiments, the platelets of the compositions are autologous platelets. In some embodiments, the PRP of the compositions provided herein is allogeneic PRP. In some embodiments, the platelets of the compositions are allogeneic platelets. Provided herein are compositions comprising placental stem cells combined with platelet rich plasma, wherein administration of the compositions to an individual in need thereof results in prolonged localization of the placental stem cells at the site of injection or implantation, relative to administration of placental stem cells not combined with platelet rich plasma. In certain embodiments, the placental stem cells are human. In other embodiments, the platelet rich plasma is human, e.g., is obtained from or derived from a human source. In other embodiments, both the placental stem cells and PRP are human.

In various embodiments, the volume to volume ratio of placental stem cells to platelet rich plasma can be between about 10:1 and 1:10.

In other embodiments, transplantation of said composition comprising placental stem cells combined with platelet rich plasma prolongs localization of the placental stem cells at the site of injection or implantation at least, or at, 1, 2, 3, 4, 5, 6, 7, 8, 9, 10, 11, 12, 13, 14, 15, 16, 17, 18, 19, 20 or 21 days post-transplant, relative to transplantation of placental stem cells not combined with platelet rich plasma. In another more specific embodiment, said composition comprising placental stem cells combined with platelet rich plasma prolongs localization of the placental stem cells at the site of injection or implantation at least, or more than 21 days post-transplant. In specific embodiments, said composition comprising placental stem cells combined with platelet rich plasma prolongs localization of the placental stem cells at the site of injection or implantation at least, or more than 25, 30, 35, 40, 45, 50, 55 weeks, or 1 year or longer post-transplant.

### 4.3 Pharmaceutical Compositions

Also provided herein are pharmaceutical compositions that comprise PRP or isolated platelets obtained as described herein, and a pharmaceutically-acceptable carrier. Further presented herein are pharmaceutical compositions of the compositions presented herein that comprise PRP or isolated platelets and, optionally, stem cells, e.g., placental stem cells,combination compositions described herein, and a pharmaceutically-acceptable carrier.

In one one embodment, for example, the PRP or isolated platelets obtained as described herein may be formulated as an injectable (see, e.g., WO 96/39101, incorporated herein by reference in its entirety) comprising a pharmaceutically acceptable carrier. In one one embodment, for example, the compositions presented herein comprising PRP or isolated platelets obtained as described herein may be formulated as an injectable (see, e.g., WO 96/39101, incorporated herein by reference in its entirety) comprising a pharmaceutically acceptable carrier. In another embodiment, the compositions presented herein may be formulated using polymerizable or cross linking hydrogels as described, e.g., in U.S. Pat. Nos. 5,709,854; 5,516,532; 5,654,381, and a pharmaceutically acceptable carrier.

In one embodiment, each component of the compositions presented herein, e.g., PRP or isolagted platelets and stem cells, e.g., placental stem cells, may be maintained prior to use, e.g., prior to administration to an individual, as separate pharmaceutical compositions to be administered sequentially or jointly to create a composition as described herein in vivo. Each component may be stored and/or used in a separate container, e.g., one bag (e.g., blood storage bag from Baxter, Becton-Dickinson, Medcep, National Hospital Products, Terumo, etc.) or separate syringe, which contains a single type of cell or cell population. In a specific embodiment, PRP or isolated platelets are contained in one bag, and stem cells, e.g., placentlal stem cells, for example placental perfusate, or placental stem cells from placental perfusate, are contained in a second bag.

In a specific embodiment, the pharmaceutical compositions may comprise one or more agents that induce cell differentiation. In certain embodiments, an agent that induces differentiation includes, but is not limited to, Ca²⁺, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyroxine, hydrocortisone, dexamethasone), sodium butyrate, TPA, DMSO, NMF, DMF, matrix elements (e.g., collagen, laminin, heparan sulfate, MATRIGEL™), or combinations thereof

In another embodiment, the pharmaceutical composition may comprise one or more agents that suppress cellular differentiation. In certain embodiments, an agent that suppresses differentiation includes, but is not limited to, human Delta-1 and human Serrate-1 polypeptides (see, Sakano et al., U.S. Pat. No. 6,337,387), leukemia inhibitory factor (LIF), stem cell factor, or combinations thereof.

The pharmaceutical compositions provided herein may, for example, be treated prior to administration to an individual with a compound that modulates the activity of TNF-α. Such compounds are disclosed in detail in, e.g., U.S. Application Publication No. 2003/0235909, which disclosure is incorporated herein in its entirety.

### 4.4 Methods of Utilizing Platelets and Platelet Rich Plasma

In particular aspects, the PRP, isolated platelets and compositions provided herein are useful in treating a disease, disorder or medical condition in an individual. For example, provided herein are methods of promoting wound healing comprising administering PRP, isolated platelets or a composition provided herein to an individual in need of wound healing. In another example, provided herein are methods of promoting promoting tissue or organ repair or regeneration, comprising administering a composition provided herein to an individual in need of tissue or organ repair or regeneration. In a particular embodiment, provided herein are methods of bone repair or regeneration comprising administering PRP, isolated platelets or a composition provided herein to an individual in need of bone repair or regeneration.

In one embodiment, presented herein are methods of promoting wound healing comprising administering PRP, isolated platelets or a composition provided herein to an individual in need of wound healing. Such methods comprise treatment of a wound, including but not limited to: an epidermal wound, skin wound, chronic wound, acute wound, external wound, internal wound, and a congenital wound (e.g., dystrophic epidermolysis bullosa). Thus, in another aspect, provided herein is a method of treating an individual having a wound, comprising administering to the individual a therapeutically-effective amount of PRP, isolated platelets or a composition as presented herein.

In other embodiments, PRP, isolated platelets or a composition provided herein is administered to an individual for the treatment of a wound infection, e.g., a wound infection followed by a breakdown of a surgical or traumatic wound. Such a wound infection can be from any microorganism known in the art, e.g., microorganisms that infect wounds originating from within the human body, which is a known reservoir for pathogenic organisms, or from environmental origin. A non-limiting example of the microorganisms, the growth of which in wounds may be reduced or prevented by the methods and compositions described herein are Staphylococcus aureus, S. epidermidis, beta haemolytic streptococci, Escherichia coli, Klebsiella and Pseudomonas species, and among the anaerobic bacteria, the Clostridium welchii or C. tartium, which are the cause of gas gangrene, mainly in deep traumatic wounds.

In other embodiments, PRP, isolated platelets or a composition provided herein is administered for the treatment of burns, including but not limited to, first-degree burns, second-degree burns (partial thickness burns), third degree burns (full thickness burns), infection of burn wounds, infection of excised and unexcised burn wounds, infection of grafted wound, infection of donor site, loss of epithelium from a previously grafted or healed burn wound or skin graft donor site, and burn wound impetigo.

In particular embodiments, PRP, isolated platelets or a composition provided herein can be used in the treatment of ulcers, e.g., leg ulcers. In various embodiments, said leg ulcer can be, for example, a venous leg ulcer, arterial leg ulcer, diabetic leg ulcer, decubitus ulcer, or split thickness skin grafted ulcer or wound. In this context, “treatment of a leg ulcer” comprises contacting the leg ulcer with an amount of PRP, isolated platelets or a composition provided herein effective to improve at least one aspect of the leg ulcer. As used herein, “aspect of the leg ulcer” includes objectively measurable parameters such as ulcer size, depth or area, degree of inflammation, ingrowth of epithelial and/or mesodermal tissue, gene expression within the ulcerated tissue that is correlated with the healing process, quality and extent of scarring etc., and subjectively measurable parameters, such as patient well-being, perception of improvement, perception of lessening of pain or discomfort associated with the ulcer, patient perception that treatment is successful, and the like.

In particular embodiments, provided herein are methods for the treatment of venous leg ulcers comprising administering PRP, isolated platelets or a composition provided herein effective to improve at least one aspect of the venous leg ulcer. Venous leg ulcers, also known as venous stasis ulcers or venous insufficiency ulcers, a type of chronic or non-healing wound, are widely prevalent in the United States, with approximately 7 million people, usually the elderly, afflicted. Worldwide, it is estimated that 1-1.3% of individuals suffer from venous leg ulcers. Approximately 70% of all leg ulcers are venous ulcers. Venous leg ulcers are often located in the distal third of the leg known as the gaiter region, and typically on the inside of the leg. The ulcer is usually painless unless infected. Venous leg ulcers typically occur because the valves connecting the superficial and deep veins fail to function properly. Failure of these valves causes blood to flow from the deep veins back out to the superficial veins. This inappropriate flow, together with the effects of gravity, causes swelling and progression to damage of lower leg tissues.

Patients with venous leg ulcers often have a history of deep vein thrombosis, leg injury, obesity, phlebitis, prior vein surgery, and lifestyles that require prolonged standing. Other factors may contribute to the chronicity of venous leg ulcers, including poor circulation, often caused by arteriosclerosis; disorders of clotting and circulation that may or may not be related to atherosclerosis; diabetes; renal (kidney) failure; hypertension (treated or untreated); lymphedema (buildup of fluid that causes swelling in the legs or feet); inflammatory diseases such as vasculitis, lupus, scleroderma or other rheumatological conditions; medical conditions such as high cholesterol, heart disease, high blood pressure, sickle cell anemia, or bowel disorders; a history of smoking (either current or past); pressure caused by lying in one position for too long; genetics (predisposition for venous disease); malignancy (tumor or cancerous mass); infections; and certain medications.

Thus, in another embodiment, provided herein is a method of treating a venous leg ulcer comprising contacting the venous leg ulcer with an amount of PRP, isolated platelets or a composition provided herein sufficient to improve at least one aspect of the venous leg ulcer. In another specific embodiment, the method additionally comprises treating an underlying cause of the venous leg ulcer.

The methods for treating a venous leg ulcer provided herein further encompass treating the venous leg ulcer by administering a therapeutically effective amount of PRP, isolated platelets or a composition provided herein, in conjunction with one or more therapies or treatments used in the course of treating a venous leg ulcer. The one or more additional therapies may be used prior to, concurrent with, or after administration of the PRP, isolated platelets or a composition provided herein. In some embodiments, the one or more additional therapies comprise compression of the leg to minimize edema or swelling. In some embodiments, compression treatments include wearing therapeutic compression stockings, multilayer compression wraps, or wrapping an ACE bandage or dressing from the toes or foot to the area below the knee.

Arterial leg ulcers are caused by an insufficiency in one or more arteries' ability to deliver blood to the lower leg, most often due to atherosclerosis. Arterial ulcers are usually found on the feet, particularly the heels or toes, and the borders of the ulcer appear as though they have been ‘punched out’. Arterial ulcers are frequently painful. This pain is relieved when the legs are lowered with feet on the floor as gravity causes more blood to flow into the legs. Arterial ulcers are usually associated with cold white or bluish, shiny feet.

The treatment of arterial leg ulcers contrasts to the treatment of venous leg ulcers in that compression is contraindicated, as compression tends to exacerbate an already-poor blood supply, and debridement is limited, if indicated at all. Thus, in another embodiment, provided herein is a method of treating an arterial leg ulcer comprising treating the underlying cause of the arterial leg ulcer, e.g., arteriosclerosis, and contacting the arterial leg ulcer with an amount of PRP, isolated platelets or a composition provided herein sufficient to improve at least one aspect of the arterial leg ulcer. In a specific embodiment, the method of treating does not comprise compression therapy.

Diabetic foot ulcers are ulcers that occur as a result of complications from diabetes. Diabetic ulcers are typically caused by the combination of small arterial blockage and nerve damage, and are most common on the foot, though they may occur in other areas affected by neuropathy and pressure. Diabetic ulcers have characteristics similar to arterial ulcers but tend to be located over pressure points such as heels, balls of the feet, tips of toes, between toes or anywhere bony prominences rub against bed sheets, socks or shoes.

Treatment of diabetic leg ulcers is generally similar to the treatment of venous leg ulcers, though generally without compression; additionally, the underlying diabetes is treated or managed. Thus, in another embodiment, provided herein is a method of treating a diabetic leg ulcer comprising treating the underlying diabetes, and contacting the diabetic leg ulcer with an amount of PRP, isolated platelets or a composition provided herein sufficient to improve at least one aspect of the diabetic leg ulcer.

Decubitus ulcers, commonly called bedsores or pressure ulcers, can range from a very mild pink coloration of the skin, which disappears in a few hours after pressure is relieved on the area to a very deep wound extending into the bone. Decubitus ulcers occur frequently with patients subject to prolonged bedrest, e.g., quadriplegics and paraplegics who suffer skin loss due to the effects of localized pressure. The resulting pressure sores exhibit dermal erosion and loss of the epidermis and skin appendages. Factors known to be associated with the development of decubitus ulcers include advanced age, immobility, poor nutrition, and incontinence. Stage 1 decubitus ulcers exhibit nonblanchable erythema of intact skin. Stage 2 decubitus ulcers exhibit superficial or partial thickness skin loss. Stage 3 decubitus ulcers exhibit full thickness skin loss with subcutaneous damage. The ulcer extends down to underlying fascia, and presents as a deep crater. Finally, stage 4 decubitus ulcers exhibit full thickness skin loss with extensive destruction, tissue necrosis, and damage to the underlying muscle, bone, tendon or joint capsule. Thus, in another embodiment, provided herein is a method of treating a decubitus leg ulcer comprising treating the underlying diabetes, and contacting the decubitus leg ulcer with an amount of PRP, isolated platelets or a composition provided herein sufficient to improve at least one aspect of the decubitus leg ulcer.

Also provided herein are methods of treating a leg ulcer by administering a composition comprising placental stem cells and platelet rich plasma in conjunction with one or more therapies or treatments used in the course of treating a leg ulcer. The one or more additional therapies may be used prior to, concurrent with, or after administration of PRP, isolated platelets or a composition provided herein. PRP, isolated platelets or a composition provided herein, and one or more additional therapies, may be used where the PRP, isolated platelets or a composition provided herein, alone, or the one or more additional therapies, alone, would be insufficient to measurably improve, maintain, or lessen the worsening of, one or more aspects of a leg ulcer.

In specific embodiments, the one or more additional therapies comprise, without limitation, treatment of the leg ulcer with a wound healing agent (e.g., PDGF, REGRANEX®); administration of an anti-inflammatory compound; administration of a pain medication; administration of an antibiotic; administration of an anti-platelet or anti-clotting medication; application of a prosthetic; application of a dressing (e.g., moist to moist dressings; hydrogels/hydrocolloids; alginate dressings; collagen-based wound dressings; antimicrobial dressings; composite dressings; synthetic skin substitutes, etc.), and the like. In another embodiment, the additional therapy comprises contacting the leg ulcer with honey. For any of the above embodiments, in a specific embodiment, the leg ulcer is a venous leg ulcer, a decubitus ulcer, a diabetic ulcer, or an arterial leg ulcer.

In another specific embodiment, the additional therapy is a pain medication. Thus, also provided herein is a method of treating a leg ulcer comprising contacting the leg ulcer with PRP, isolated platelets or a composition provided herein, and administering a pain medication to lessen or eliminate leg ulcer pain. In a specific embodiment, the pain medication is a topical pain medication.

In another specific embodiment, the additional therapy is an anti-infective agent. In one embodiment, the anti-infective agent is one that is not cytotoxic to healthy tissues surrounding and underlying the leg ulcer; thus, compounds such as iodine and bleach are disfavored. Thus, treatment of the leg ulcer, in one embodiment, comprises contacting the leg ulcer with PRP, isolated platelets or a composition provided herein, and administering an anti-infective agent. The anti-infective agent may be administered by any route, e.g., topically, orally, buccally, intravenously, intramuscularly, anally, etc. In a specific example, the anti-infective agent is an antibiotic, a bacteriostatic agent, antiviral compound, a virustatic agent, antifungal compound, a fungistatic agent, or an antimicrobial compound. In another specific embodiment, the anti-infective agent is ionic silver. In a more specific embodiment, the ionic silver is contained within a hydrogel. In specific embodiments, the leg ulcer is a venous leg ulcer, arterial leg ulcer, decubitus ulcer, or diabetic ulcer.

In another specific embodiment of the methods of treatment described herein, PRP, isolated platelets or a composition provided herein is used for the treatment of orthopedic defects, including but not limited to, bone defects, disc herniation and degenerative disc disease. Thus, in another aspect, provided herein is a method of treating an individual having a bone defect, disc herniation, or degenerative disc disease, comprising administering to the individual a therapeutically-effective amount of PRP, isolated platelets or a composition provided herein.

In a particular aspect, provided herein is a method for treating a bone defect in a subject, comprising administering to a subject in need thereof a therapeutically effective amount of an implantable or injectable composition as described herein sufficient to treat the bone defect in the subject. In certain embodiments, the bone defect is an osteolytic lesion associated with a cancer, a bone fracture, or a spine, e.g., in need of fusion. In certain embodiments, the osteolytic lesion is associated with multiple myeloma, bone cancer, or metastatic cancer. In certain embodiments, an implantable composition is administered to the subject. In certain embodiments, an implantable composition is surgically implanted, e.g., at the site of the bone defect. In certain embodiments, an injectable composition is administered to the subject. In certain embodiments, an injectable composition is surgically administered to the region of the bone defect.

In particular, presented herein are methods for treatment of herniated discs and degenerative disc disease comprising administration of PRP, isolated platelets or a composition provided herein. In some embodiments, the degenerative disc disease is characterized on x-ray tests or MRI scanning of the spine as a narrowing of the normal “disc space” between the adjacent vertebrae.

Disc degeneration, medically referred to as spondylosis, can occur with age when the water and protein content of the cartilage of the body changes. This change results in weaker, more fragile and thin cartilage. Because both the discs and the joints that stack the vertebrae (facet joints) are partly composed of cartilage, these areas are subject to degenerative changes, which renders the disc tissue susceptible to herniation. The gradual deterioration of the disc between the vertebrae is referred to as degenerative disc disease. Degeneration of the disc can cause local pain in the affected area, for example, radiculopathy, i.e., nerve irritation caused by damage to the disc between the vertebrae. In particular, weakness of the outer ring leads to disc bulging and herniation. As a result, the central softer portion of the disc can rupture through the outer ring of the disc and abut the spinal cord or its nerves as they exit the bony spinal column.

Any level of the spine can be affected by disc degeneration. Thus, in some embodiments, the degenerative disc disease treatable by the methods provided herein is cervical disc disease, i.e., disc degeneration that affects the spine of the neck, often accompanied by painful burning or tingling sensations in the arms. In some embodiments, the degenerative disc disease is thoracic disc disease, i.e., disc degeneration that affects the mid-back. In some embodiments, the degenerative disc disease is lumbago, i.e., disc degeneration that affects the lumbar spine.

In particular embodiments, the method for treating degenerative disc disease in a subject comprises administering to a subject in need thereof a therapeutically effective amount of an implantable or injectable composition described herein sufficient to treat cervical or lumbar radiculopathy in the subject. In some embodiments, the lumbar radiculopathy is accompanied by incontinence of the bladder and/or bowels. In some embodiments, the method for treating degenerative disc disease in a subject comprises administering to a subject in need thereof a therapeutically effective amount of an implantable or injectable composition described herein sufficient to relieve sciatic pain in the subject.

In some embodiments of the methods of treating disc degeneration in an individual with PRP, isolated platelets or a composition provided herein, wherein the disc degeneration of the individual occurs at the intervertebral disc between C1 and C2; between C2 and C3; between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

In some embodiments of the methods of treating disc herniation in an individual with PRP, isolated platelets or a composition provided herein, wherein the disc herniation occurs at the intervertebral disc between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

Degenerative arthritis (osteoarthritis) of the facet joints is also a cause of localized lumbar pain that can be detected with plain x-ray testing. Wear of the facet cartilage and the bony changes of the adjacent joint is referred to as degenerative facet joint disease or osteoarthritis of the spine.

The methods for treating degerative disc disease provided herein further encompass treating degerative disc disease by administering a therapeutically effective amount of PRP, isolated platelets or a composition provided herein, in conjunction with one or more therapies or treatments used in the course of treating degerative disc disease. The one or more additional therapies may be used prior to, concurrent with, or after administration of PRP, isolated platelets or a composition provided herein. In some embodiments, the one or more additional therapies comprise administration of medications to relieve pain and muscles spasm, cortisone injection around the spinal cord (epidural injection), physical therapy (heat, massage, ultrasound, electrical stimulation), and rest (not strict bed rest, but avoiding re-injury).

In some embodiments, the one or more additional therapies comprise operative intervention, for example, where the subject presents with unrelenting pain, severe impairment of function, or incontinence (which can indicate spinal cord irritation). In some embodiments, the operative intervention comprises removal of the herniated disc with laminotomy (producing a small hole in the bone of the spine surrounding the spinal cord), laminectomy (removal of the bony wall adjacent to the nerve tissues), by needle technique through the skin (percutaneous discectomy), disc-dissolving procedures (chemonucleolysis), and others.

### Equivalents

The compositions and methods disclosed herein are not to be limited in scope by the specific embodiments described herein. Indeed, various modifications of the compositions and methods in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method for isolation of platelets from umbilical cord blood or blood obtained from placenta, the method comprising: removing erythrocytes from the umbilical cord blood or blood obtained from placenta to produce plasma, and separating leukocytes from the plasma.

2. The method of claim 1, wherein the erythrocytes are removed by centrifugation of the blood.

3. The method of claim 2, wherein the erythrocytes are removed by introducing a plasma volume expander into the blood.

4. The method of claim 3, wherein erythrocytes are spontaneously sedimented following introduction of the plasma volume expander into the blood.

5. The method of claim 3, wherein erythrocytes are sedimented by centrifugation following introduction of the plasma volume expander into the blood.

6. The method of claim 3, wherein the plasma volume expander is hetastarch or pentastarch.

7. The method of claim 1, wherein the plasma is centrifuged for a time sufficient to separate leukocytes from platelets in the plasma, thereby producing platelet rich plasma (PRP).

8. The method of claim 7, wherein the plasma is centrifuged at about 200×G to about 500×G.

9. The method of claim 7, wherein the plasma is centrifuged at about 300×G to about 400×G.

10. The method of claim 7, wherein the plasma is centrifuged for about 5 to about 30 minutes.

11. (canceled)

12. (canceled)

13. (canceled)

14. A method for isolation of platelets from umbilical cord blood or blood obtained from a placenta, the method comprising: centrifuging the blood at about 100×G to about 500×G for about 10 minutes to about 30 minutes, thereby producing PRP.

15. The method of claim 14, comprising centrifuging the blood at about 100×G to about 200×G.

16. The method of claim 14, comprising centrifuging the blood for about 20 to about 25 minutes.

17. The method of claim 7, further comprising buffering the PRP.

18. The method of claim 7, further comprising cryopreserving the PRP.

19. The method of claim 18, wherein cryopreserving the PRP comprises freezing the PRP.

20. The method of claim 18, wherein cryopreserving the PRP comprises freeze drying the PRP.

21. The method of 20, further comprising storing the freeze dried PRP at room temperature under vacuum.

22. The method of claim 7, further comprising: centrifuging the PRP at about 500×G to about 4000×G for about 20 to about 60 minutes to pellet the platelets, and removing the resulting supernatant.

23. The method of claim 22, comprising centrifuging the PRP at about 2000×G to about 4000×G for about 10 to about 20 minutes, to pellet the platelets, and removing the resulting supernatant.

24. (canceled)

25. The method of claim 22, further comprising resuspending the platelets in a buffer.

26. The method of claim 22, further comprising cryopreserving the platelets.

27. The method of claim 26, wherein cryopreserving the platelets comprises freezing the platelets.

28. The method of claim 26, wherein cryopreserving the platelets comprises freeze drying the platelets.

29. The method of 28, further comprising storing the freeze dried platelets at room temperature under vacuum.

30. (canceled)

31. The method of claim 1, wherein the cord blood is human cord blood.

32. (canceled)

33. The method of claim 1, wherein the blood is from human placenta.

34-37. (canceled)
